(19) United States
(12) Patent Application Publication
(10) Pub. No.: US 2024/0013914 A1
(43) Pub. Date: Jan. 11, 2024

(54) DETERMINATION APPARATUS

(71) Applicant: NEC Corporation, Minato-ku, Tokyo (JP)

(72) Inventors: Yuji OHNO, Tokyo (JP); Toshinori HOSOI, Tokyo (JP); Masahiro KUBO, Tokyo (JP)

(73) Assignee: NEC Corporation, Minato-ku, Tokyo (JP)

(21) Appl. No.: 18/025,466

(22) PCT Filed: Sep. 17, 2020
(86) PCT No.: PCT/JP2020/035295

§ 371 Date: Mar. 9, 2023

(51) Int. Cl. G16H 50/20; G16H 50/30; A61B 5/11; A61B 5/0205

(52) U.S. Cl. CPC G16H 50/20; G16H 50/30; A61B 5/1126; A61B 5/02055

(57) ABSTRACT

A determination apparatus 600 includes an agitation state determination unit 621 configured to determine an agitation state of a target based on acquired information, a condition determination unit 622 configured to determine a condition of the target based on the acquired information, and a specifying unit 623 configured to specify which to perform, the determination processing performed by the agitation state determination unit 621 or the determination processing performed by the condition determination unit 622, according to an acquisition condition of the information.

### TECHNICAL FIELD

The present invention relates to a determination apparatus, a determination method, and a recording medium.

### BACKGROUND ART

When a patient is in an agitated state, a risk of extubation, needle dislodgement, decannulation, tumble, a fall, and the like increases, and the patient may be, for example, injured as a result thereof. In this regard, there have been known techniques for determining a predictive sign of agitation to reduce such a risk and a possibility.

Examples of known documents discussing the techniques for determining a predictive sign of agitation include Patent Literature 1. Patent Literature 1 discusses a biological information processing system including a determination unit and an estimation unit. According to Patent Literature 1, the determination unit determines discrimination information indicating whether or not a condition of a patient has changed in comparison with a normal state based on features of biological information of the patient. Then, the estimation unit estimates countermeasure information for the patient based on the discrimination information determined by the determination unit and countermeasure prediction parameters learned in advance. Further, Patent Literature 1 discloses a heartbeat and the like as one example of the biological information, and discloses an agitation score indicating a possibility that the patient is in an agitated state as one example of the discrimination information.

### CITATION LIST

### Patent Literature

Patent Literature 1: International Publication No. 2019/073927

### SUMMARY OF INVENTION

### Technical Problem

In the case of the technique discussed in Patent Literature 1, the estimation and the determination are made based on the features of the biological information acquired around the bed. Therefore, it is difficult to appropriately determine the state of the patient when, for example, the patient leaves the bed.

Under these circumstances, one of objects of the present invention is to provide a determination apparatus, a determination method, and a recording medium capable of solving the problem that it is difficult to appropriately determine the state of the patient.

### Solution to Problem

To achieve the above-described object, according to one aspect of the present disclosure, a determination apparatus is configured to include
an agitation state determination unit configured to determine an agitation state of a target based on acquired information,
a condition determination unit configured to determine a condition of the target based on the acquired information, and
a specifying unit configured to specify which to perform, the determination processing performed by the agitation state determination unit or the determination processing performed by the condition determination unit, according to an acquisition condition of the information.

Further, according to another aspect of the present disclosure, a determination method is configured to cause a computer to
acquire information, and
specify which to perform, determination processing for determining an agitation state of a target or determination processing for determining a condition of the target, according to an acquisition condition of the information.

Further, according to another aspect of the present disclosure, a recording medium records a program for causing a computer to realize processing including
acquiring information, and
specifying which to perform, determination processing for determining an agitation state of a target or determination processing for determining a condition of the target, according to an acquisition condition of the information.

### Advantageous Effects of Invention

According to each of the above-described configurations, the state of the patient can be appropriately determined.

### BRIEF DESCRIPTION OF DRAWINGS

FIG. 1 is a diagram illustrating an example of the overall configuration of a determination system according to a first exemplary embodiment of the present disclosure.

FIG. 2 is a block diagram illustrating an example of the configuration of a sensor apparatus illustrated in FIG. 1.

FIG. 3 is a block diagram illustrating an example of the configuration of a bed terminal illustrated in FIG. 1.

FIG. 4 is a block diagram illustrating an example of the configuration of a relay apparatus illustrated in FIG. 1.

FIG. 5 is a block diagram illustrating an example of the configuration of a determination apparatus illustrated in FIG. 1.

FIG. 6 is a diagram illustrating one example of information stored in a storage unit.

FIG. 7 is a diagram illustrating one example of vital data included in sensing data illustrated in FIG. 5.

FIG. 8 is a diagram illustrating one example of a vital score included in score information illustrated in FIG. 1.

FIG. 9 is a diagram for illustrating an example of processing by an agitation state determination unit.

FIG. 10 is a flowchart illustrating examples of operations of the determination apparatus.

FIG. 11 is a block diagram illustrating another example of the configuration of the determination apparatus.

FIG. 12 is a diagram illustrating the hardware configuration of a determination apparatus according to a second exemplary embodiment of the present disclosure.

FIG. 13 is a block diagram illustrating an example of the configuration of the determination apparatus.

### DESCRIPTION OF EMBODIMENTS

### First Exemplary Embodiment

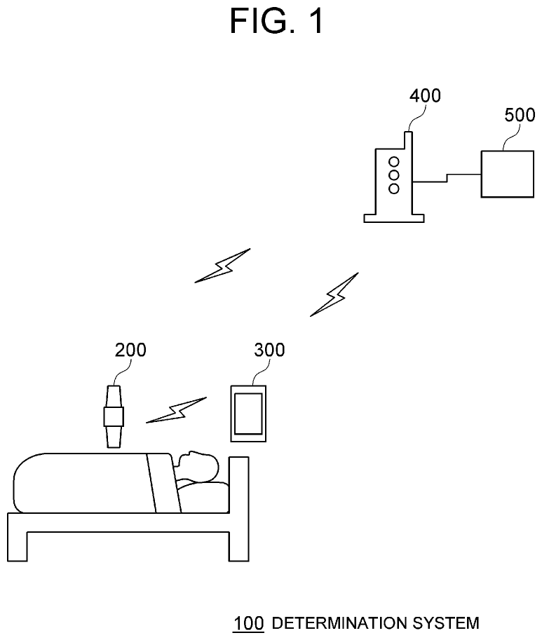

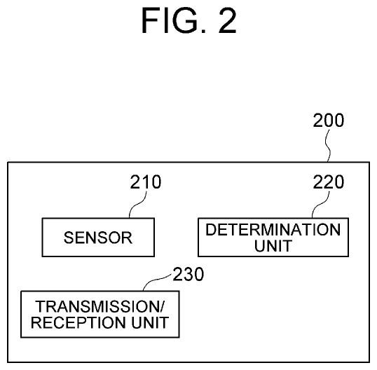

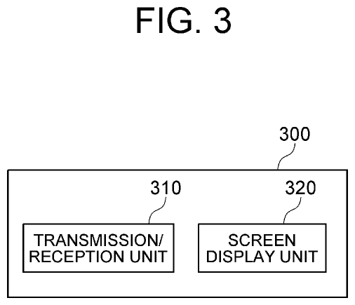

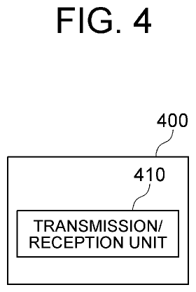

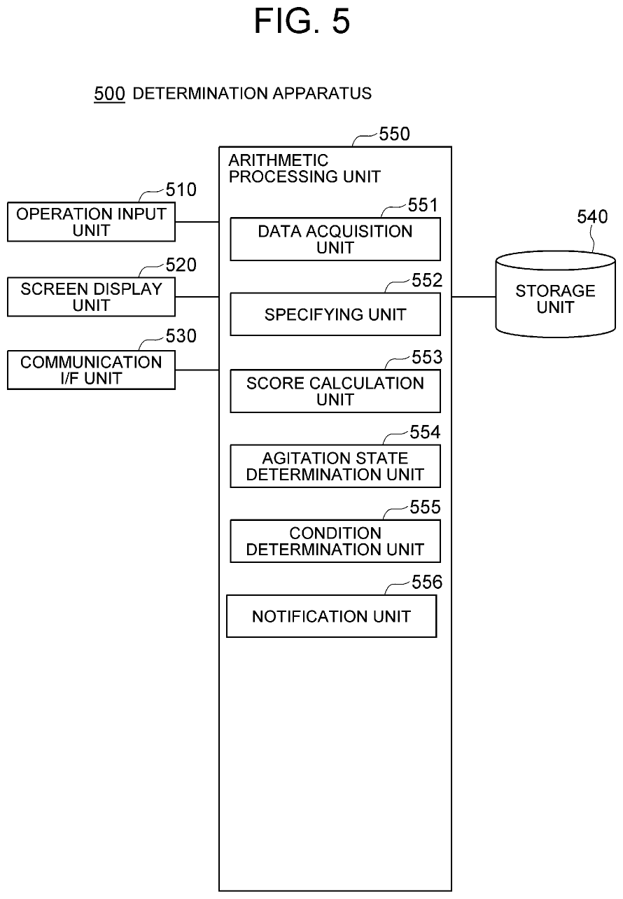

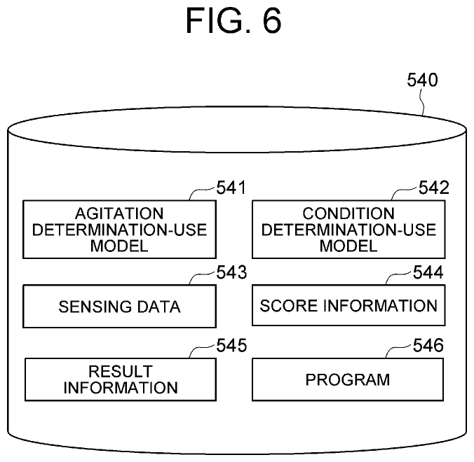

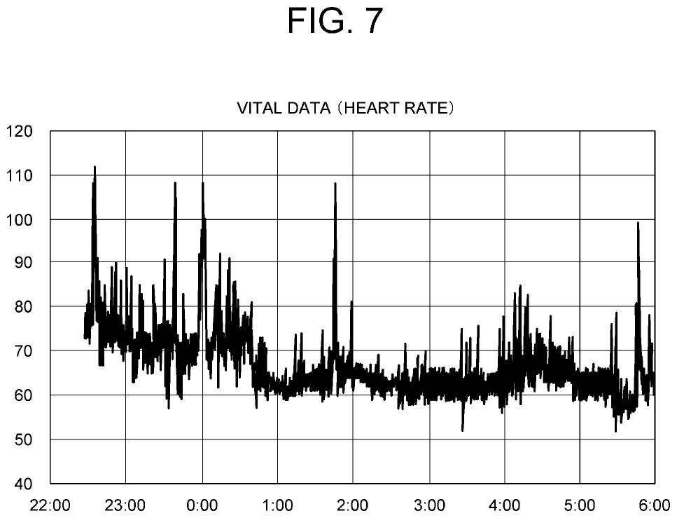

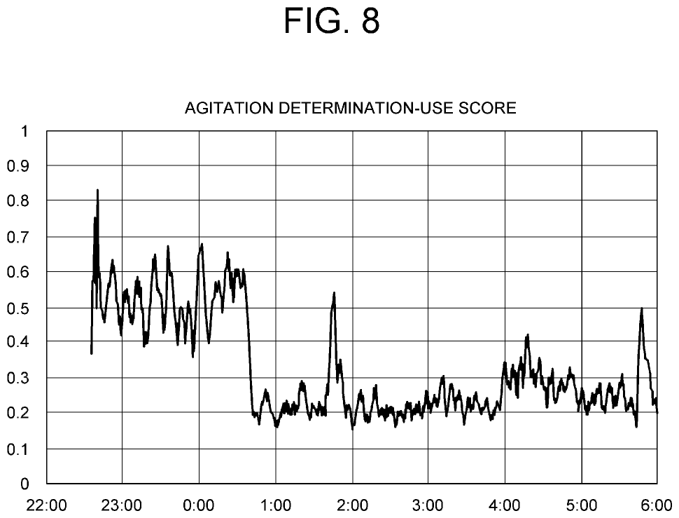

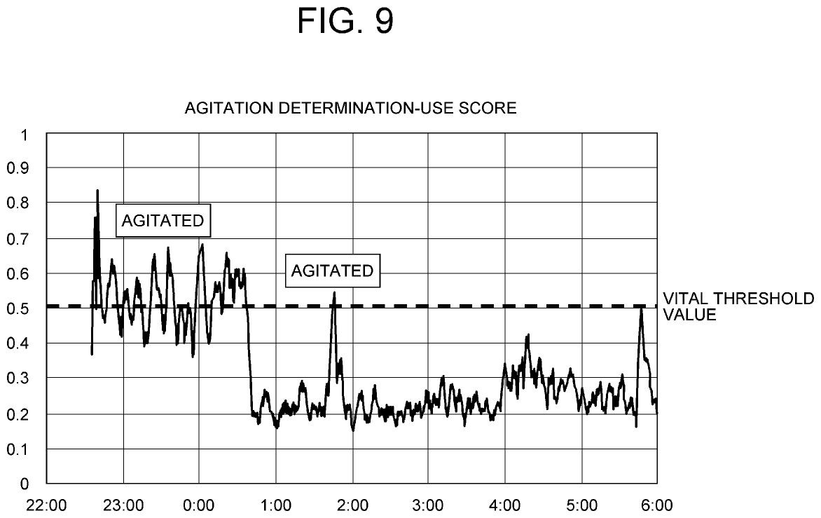

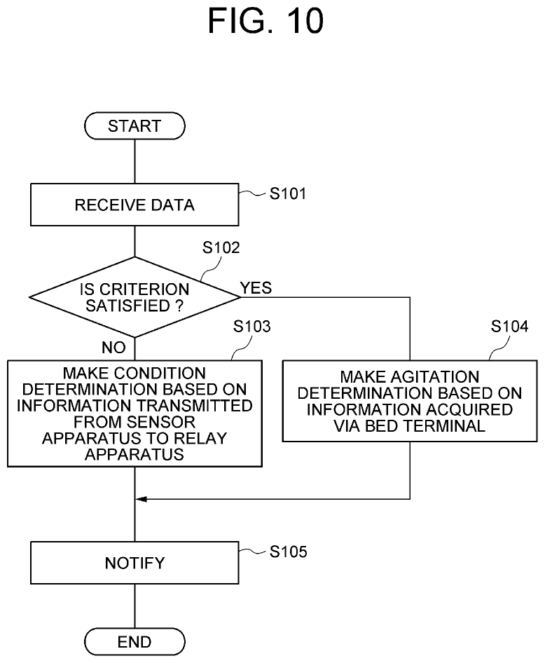

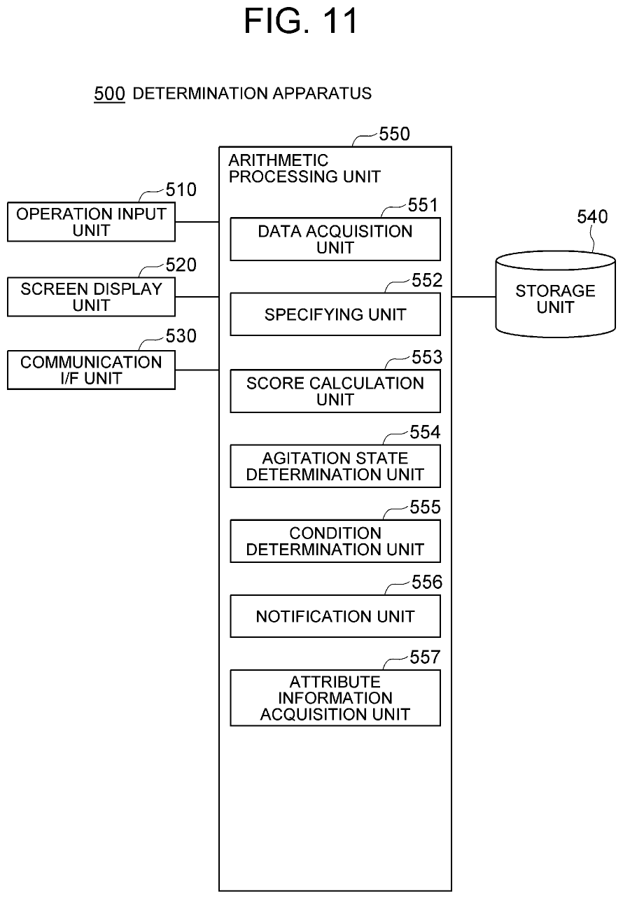

A first exemplary embodiment of the present disclosure will be described with reference to FIGS. 1 to 11. FIG. 1 is a diagram illustrating an example of the overall configuration of a determination system 100. FIG. 2 is a block diagram illustrating an example of the configuration of a sensor apparatus 200. FIG. 3 is a block diagram illustrating an example of the configuration of a bed terminal 300. FIG. 4 is a block diagram illustrating an example of the configuration of a relay apparatus 400. FIG. 5 is a block diagram illustrating an example of the configuration of a determination apparatus 500. FIG. 6 is a diagram illustrating one example of information stored in a storage unit 540. FIG. 7 is a diagram illustrating one example of vital data. FIG. 8 is a diagram illustrating one example of a vital score included in score information 544. FIG. 9 is a diagram for illustrating an example of processing by an agitation state determination unit 554. FIG. 10 is a flowchart illustrating examples of operations of the determination apparatus 500. FIG. 11 is a block diagram illustrating another example of the configuration of the determination apparatus 500.

The first exemplary embodiment of the present disclosure will be described regarding the determination system 100, which determines a state of a patient. The determination system 100 according to the present exemplary embodiment includes the agitation state determination unit 554, which determines an agitation state of the patient, and a condition determination unit 555, which determines a condition of the patient. As will be described below, the determination system 100 specifies which to perform, the determination processing performed by the agitation state determination unit 554 and the determination processing performed by the condition determination unit 555 according to an acquisition condition of information that the agitation state determination unit 554 or the condition determination unit 555 uses when performing the determination processing. By performing the determination processing according to such a specified selection, the determination system 100 can make an appropriate determination according to, for example, whether the patient leaves a predetermined location.

The determination system 100, which will be described in the present exemplary embodiment, can be used in a scene such as an acute hospital, a convalescent hospital, a care facility, or monitoring at home. The determination system 100 may be used in a scene different from the above-described examples.

In the present exemplary embodiment, the agitation refers to a state in which the patient is restless and upset. The agitation may be caused by delirium or the like. Further, the agitation state indicates a state regarding the agitation of the patient. The agitation state indicates, for example, whether the patient is agitated or whether the patient exhibits a predictive sign of agitation. Note that the agitation state may include another index regarding a likelihood of the patient's agitation. When the patient is agitated, the patient may conduct a problematic behavior, such as falling off the bed, removing intubation, shouting abnormally, or committing violence.

Further, in the present exemplary embodiment, the condition of the patient determined by the condition determination unit 555 is, for example, a concept wider than the agitation state. For example, the condition of the patient can include at least one of a positional state, a motion state, a vital state, a problem state, or the like. Now, the positional state refers to a state according to the position where the patient stays, such as a state that the patient is present at an unpermitted place or a state that the patient is moving in an unpermitted direction. Further, the motion state refers to a state according to the motion of the patient, such as a state that the patient is standing or a state that the patient is walking. Further, the vital state refers to a state according to the vital of the patient, such as the heart rate and/or the perspiration rate of the patient, or whether a problem has occurred with the heart rate or the perspiration rate. Further, the problem state refers to a state according to whether a problem has occurred with the patient, such as a state that the patient has fallen down, a state that the patient is straying, a state that the patient is moving in an abnormal direction, or a state that the patient is wandering around. Note that the problem state may be determined based on at least one of the positional state, the motion state, or the vital state. For example, a problem state that a problem has occurred with the patient is determined based on a vital state that the heart rate of the patient is abnormal. Further, for example, a detailed problem state that the patient has fallen down at a predetermined place is determined based on a positional state that the patient is present at the unpermitted predetermined place, a motion state that the patient remains still, and a vital state that the heart rate of the patient is abnormal. The positional state, the motion state, the vital state, and the problem state may be different from the above-described examples. Note that the condition of the patient may include the agitation state or may not include the agitation state.

FIG. 1 illustrates an example of the configuration of the determination system 100. Referring to FIG. 1, the determination system 100 includes, for example, the sensor apparatus 200, the bed terminal 300, the relay apparatus 400, and the determination apparatus 500. As illustrated in FIG. 1, the sensor apparatus 200 and the bed terminal 300, the sensor apparatus 200 and the relay apparatus 400, the bed terminal 300 and the relay apparatus 400, and the relay apparatus 400 and the determination apparatus 500 are mutually communicably connected via wired or wireless communication for each of them. For example, the connection between the sensor apparatus 200 and the bed terminal 300 is realized by a power-saving method compared to the connection between the bed terminal 300 and the relay apparatus 400. For example, the connection between the sensor apparatus 200 and the bed terminal 300 is established using power-saving wireless communication such as Bluetooth (registered trademark), and the connection between the bed terminal 300 and the relay apparatus 400 is established using wireless communication such as Wi-Fi (registered trademark). Further, the connection between the sensor apparatus 200 and the relay apparatus 400 is realized by a method that supports further long-range communication compared to the connection between the sensor apparatus 200 and the bed terminal 300. For example, the connection between the sensor apparatus 200 and the bed terminal 300 is established using power-saving wireless communication such as Bluetooth, and the connection between the sensor apparatus 200 and the relay apparatus 400 is established using wireless communication such as Wi-Fi. Note that the sensor apparatus 200 and the relay apparatus 400 do not have to be constantly communicably connected. In other words, the determination system 100 may be configured in such a manner that the communicable connection is established between the sensor apparatus 200 and the relay apparatus 400 as need arises, such as breakage of the connection between the sensor apparatus 200 and the bed terminal 300.

Note that the number of sensor apparatuses 200, the number of bed terminals 300, the number of relay apparatuses 400, and the number of determination apparatuses 500 included in the determination system 100 are not limited to the numbers exemplarily illustrated in FIG. 1. For example, the determination system 100 can include a plurality of sensor apparatuses 200, a plurality of bed terminals 300, a plurality of relay apparatuses 400, and/or a plurality of determination apparatuses 500.

The sensor apparatus 200 measures information required for the determination processing such as the agitation determination and the condition determination, such as vital data and/or positional data of the patient set as a target. Further, the sensor apparatus 200 can transmit the information acquired from the measurement to the determination apparatus 500 via a plurality of routes (i.e., different routes). For example, in the case of the present exemplary embodiment, the sensor apparatus 200 is a wearable sensor, such as a smartwatch worn by the patient. The sensor apparatus 200 may be a fixed-type sensor fixed to a camera or a bed set up at a predetermined place, or an installed-type sensor installed around the bed, or the like without being limited to the example in which the sensor apparatus 200 is a wearable sensor.

FIG. 2 illustrates an example of the configuration of the sensor apparatus 200. Referring to FIG. 2, the sensor apparatus 200 includes, for example, a sensor 210, a determination unit 220, and a transmission/reception unit 230. For example, the sensor apparatus 200 can realize each of the above-described processing units by hardware. The sensor apparatus 200 may realize each of the above-described processing units through execution of a program stored in a storage device by an arithmetic device such as a CPU.

The sensor 210 is a device that acquires information for detecting the state/condition and/or the motion of the patient. The sensor 210 is at least one of, for example, a vital sensor, a body motion sensor, a GPS (Global Positioning System) sensor, or a camera. Then, the vital sensor is at least one of, for example, a heart rate sensor, a respiratory rate sensor, a blood pressure sensor, a body temperature sensor, or a blood oxygen saturation level sensor. Further, the body motion sensor is at least one of a speed sensor, a gyro sensor (an angular velocity sensor), an angular sensor, a microphone, or the like.

The sensor 210 acquires the information for detecting the state/condition and/or the motion of the patient using the configuration like the above-described example. The information for detecting the state/condition and/or the motion of the patient includes at least one of the vital data, the body motion data, or the positional information indicating the position of the patient. Note that the vital data refers to a physical amount varying according to the vital activity of the patient. The vital data includes, for example, a heart rate, a respiratory rate, a blood pressure value, a body temperature, a skin temperature, a blood flow rate, and/or a blood oxygen saturation level of the patient. Further, the body motion data refers to a physical amount regarding the motion of the patient's body. For example, the body motion data includes an acceleration, an angular velocity, and/or an angle at a predetermined portion such as the patient's arm, body, or foot, a vocal volume, and/or the like. Note that, in the case where the sensor 210 is a camera, the sensor 210 may acquire a video or an image as the information for detecting the state/condition and/or motion of the patient and acquire at least one of the vital data, the body motion data, or the positional information from the video or the image.

The determination unit 220 determines an apparatus to which the information acquired by the sensor 210 is transmitted. In other words, the determination unit 220 determines a route used when the information is transmitted to the determination apparatus 500. For example, the determination unit 220 determines which apparatus is the destination to which the information acquired by the sensor 210 is transmitted, the bed terminal 300 or the relay apparatus 400.

As one example, the determination unit 220 determines to transmit the information to the bed terminal 300 while the connection is established between the sensor apparatus 200 and the bed terminal 300 via the power-saving wireless communication. The determination unit 220 may determine to transmit the information to the bed terminal 300 and not to transmit the information to the relay apparatus 400 while the communication is established between the sensor apparatus 200 and the bed terminal 300. On the other hand, the determination unit 220 determines to transmit the information to the relay apparatus 400 using the wireless communication when the connection between the sensor apparatus 200 and the bed terminal 300 via the power-saving wireless communication is broken. The determination unit 220 may determine to establish a connection between the sensor apparatus 200 and the relay apparatus 400 and transmit the information to the relay apparatus 400 using the wireless communication when the connection between the sensor apparatus 200 and the bed terminal 300 via the power-saving wireless communication is broken.

The determination unit 220 determines to which apparatus the information is transmitted, the bed terminal 300 or the relay apparatus 400, based on the communication condition between the sensor apparatus 200 and the bed terminal 300 in this manner by way of example. Note that the determination unit 220 may determine to transmit the information to the bed terminal 300 and also transmit the information to the relay apparatus 400 while the connection is established between the sensor apparatus 200 and the bed terminal 300 via the power-saving wireless communication.

Note that the determination processing by the determination unit 220 is not limited to the above-described example. For example, the determination unit 220 can make the above-described determination using the positional information acquired by the sensor 210. For example, the determination unit 220 confirms whether the patient leaves the predetermined location based on the positional information. Note that the predetermined location is a location where the patient should stay or a location where the patient mainly stays, and is, for example, a location on the bed of the patient. Then, if confirming that the patient does not leave the predetermined location, the determination unit 220 determines to transmit the data to the bed terminal 300. On the other hand, if confirming that the patient leaves the predetermined location, the determination unit 220 determines to transmit the data to the relay apparatus 400. The determination unit 220 may be configured to make the determination based on the positional information in this manner. Note that the confirmation about whether the patient leaves the predetermined location may be confirmation about whether the patient stays within a predetermined range from the predetermined location. The predetermined range from the predetermined location is, for example, the vicinity of the bed or the hospital room. Further, the determination unit 220 may make the determination based on a combination of the communication condition between the sensor apparatus 200 and the bed terminal 300 and the positional information. The determination unit 220 may make the determination using a method different from the above-described examples.

Further, the determination unit 220 may select information to transmit along with determining the apparatus to which the information is transmitted. For example, the determination unit 220 can select information to transmit according to the determined apparatus to which the information is transmitted. For example, if determining to transmit the information to the bed terminal 300, the determination unit 220 selects information required to make the agitation determination among the pieces of information for detecting the state/condition and/or the motion of the patient that are acquired by the sensor 210. More specifically, the determination unit 220 selects the vital data and/or the body motion data as the information to transmit among the pieces of information for detecting the state/condition and/or the motion of the patient that are acquired by the sensor 210. On the other hand, if determining to transmit the information to the relay apparatus 400, the determination unit 220 selects information required to make the condition determination among the pieces of information for detecting the state/condition and/or the motion of the patient that are acquired by the sensor 210. More specifically, the determination unit 220 selects the positional information and/or the like as the information to transmit among the pieces of information for detecting the state/condition and/or the motion of the patient that are acquired by the sensor 210. The determination unit 220 can select the information to transmit according to the transmission destination in this manner by way of example. According to such a configuration, the required information can be correctly transmitted at each timing. Further, power consumption can be saved because unnecessary information is not transmitted. Note that the determination unit 220 may make a selection different from the above-described examples, such as selecting the vital data and/or the like as the information to transmit even when determining to transmit the information to the relay apparatus 400. Further, the determination unit 220 may determine to transmit information into which the information acquired by the sensor 210 is processed by, for example, thinning out the vital data and/or the like acquired by the sensor 210 at a predetermined time interval, when transmitting the vital data and/or the like to the relay apparatus 400. In other words, the determination unit 220 may determine to transmit information processed in such a manner that the information to transmit to the relay apparatus 400 is adjusted into low-volume information compared to information to transmit to the bed terminal 300. Transmitting the information processed in such a manner can contribute to suppressing an increase in the power consumption when the Wi-Fi connection is established. Note that the determination unit 220 may be configured to determine to always thin out the information to transmit to the relay apparatus 400.

The transmission/reception unit 230 includes an antenna or the like. The transmission/reception unit 230 transmits and receives the information between the sensor apparatus 200, and the bed terminal 300 and the relay apparatus 400 according to the result of the determination made by the determination unit 220. In other words, the transmission/reception unit 230 transmits the information acquired by the sensor apparatus 200 or the like to the information transmission destination determined by the determination unit 220. Note that, if the determination unit 220 selects the information to transmit, the transmission/reception unit 230 transmits the information selected by the determination unit 220 to the information transmission destination determined by the determination unit 220.

For example, if the determination unit 220 determines to transmit the information to the bed terminal 300, the transmission/reception unit 230 transmits the information acquired by the sensor 210 or the information selected by the determination unit 220, the identification information of the sensor apparatus 200, and the like to the bed terminal 300. On the other hand, if the determination unit 220 determines to transmit the information to the relay apparatus 400, the transmission/reception unit 230 transmits the information acquired by the sensor 210 or the information selected by the determination unit 220, the identification information of the sensor apparatus 200, and the like to the relay apparatus 400.

The transmission/reception unit 230 transmits the information according to the result of the selection made by the determination unit 220 to the transmission destination according to the determination made by the determination unit 220 in this manner by way of example.

Note that the determination unit 220 may determine not to transmit the information to the relay apparatus 400 while the communication is established between the sensor apparatus 200 and the bed terminal 300. Configuring the determination unit 220 in this manner means that the transmission/reception unit 230 transmits the information to only any one of the bed terminal 300 and the relay apparatus 400. In other words, it can also be stated that the transmission/reception unit 230 may be configured to transmit the information to only any one of the bed terminal 300 and the relay apparatus 400.

This is an example of the configuration of the sensor apparatus 200.

Note that the sensor apparatus 200 may be configured of one device or may be configured of a plurality of devices. For example, the sensor apparatus 200 can be configured of one device having functions of the vital sensor, the body motion sensor, and the like. Alternatively, the sensor apparatus 200 may be realized by mutually communicably connecting a plurality of devices, such as a device having a function as the vital sensor, a device having a function as the body motion sensor, and a device having functions as the transmission/reception unit 230 and the determination unit 240. Note that, in the case where the sensor apparatus 200 is configured of a plurality of devices, the plurality of devices may each include a transmission/reception unit.

The bed terminal 300 is an information processing apparatus set up in advance at, for example, a predetermined location such as around the bed on which the patient stays. The bed terminal 300, for example, transfers data. The bed terminal 300 is, for example, a smartphone, and has a screen display function. The bed terminal 300 may be an apparatus different from the smartphone. Note that the bed terminal 300 is a terminal set up at a predetermined location where the patient should stay or a predetermined location based on which a range where the patient should stay is defined, and is not limited to the terminal set up around the bed.

FIG. 3 illustrates an example of the configuration of the bed terminal 300. Referring to FIG. 3, the bed terminal 300 includes, for example, a transmission/reception unit 310 and a screen display unit 320. For example, the bed terminal 300 can realize each of the above-described processing units by hardware. The bed terminal 300 may realize each of the above-described processing units through execution of a program stored in a storage device by an arithmetic device such as a CPU.

The transmission/reception unit 310 includes an antenna or the like, and transmits and receives data between the bed terminal 300 and the sensor apparatus 200 using the power-saving wireless communication and also transmits and receives data between the bed terminal 300 and the relay apparatus 400 using the wireless communication. For example, the transmission/reception unit 310 receives information transmitted from the sensor apparatus 200. Then, the transmission/reception unit 310 transmits, for example, the information received from the sensor apparatus 200 to the relay apparatus 400.

Further, the transmission/reception unit 310 can transmit information according to a communication condition between the bed terminal 300 and the sensor apparatus 200 to the relay apparatus 400. For example, the transmission/reception unit 310 can transmit the information according to a communication condition, such as a condition that communication is established between the bed terminal 300 and the sensor apparatus 200 or a condition that communication between the bed terminal 300 and the sensor apparatus 200 is broken, to the relay apparatus 400.

Further, the transmission/reception unit 310 can receive information indicating the result of the agitation determination or the condition determination from the determination apparatus 500 via the relay apparatus 400.

The screen display unit 320 displays the information that the transmission/reception unit 310 receives from the sensor apparatus 200, the information indicating the result of the agitation determination or the condition determination that is received from the determination apparatus 500 via the relay apparatus 400, and the like on a screen.

The relay apparatus 400 is an apparatus that transfers data. The relay apparatus 400 is set up in advance, for example, around the location where the determination apparatus 500 is set up or at a position where data can be received from the plurality of bed terminals 300. The relay apparatus 400 is, for example, a wireless base station. The relay apparatus 400 may be an apparatus different from the wireless base station. Note that the present exemplary embodiment is described assuming that the relay apparatus 400 and the determination apparatus 500 are different configurations by way of example. However, the relay apparatus 400 and the determination apparatus 500 may be realized by the same terminal. In other words, the configuration of the relay apparatus 400 and the configuration of the determination apparatus 500 may be realized in the same apparatus. Further, the determination system 100 does not have to include the relay apparatus 400. In other words, information may be transmitted and received directly between the bed terminal 300 and the determination apparatus 500 and between the sensor apparatus 200 and the determination apparatus 500. In this case, the bed terminal 300 may realize the configuration of the determination apparatus 500.

FIG. 4 illustrates an example of the configuration of the relay apparatus 400. Referring to FIG. 4, the relay apparatus 400 includes, for example, a transmission/reception unit 410. For example, the relay apparatus 400 can realize the above-described processing unit by hardware. The relay apparatus 400 may realize the above-described processing unit through execution of a program stored in a storage device by an arithmetic device such as a CPU.

The transmission/reception unit 410 includes an antenna or the like, and transmits and receives data between the sensor apparatus 200, the bed terminal 300, the determination apparatus 500, and the like using wireless communication. The transmission/reception unit 410 receives, for example, information transmitted from the sensor apparatus 200 or the bed terminal 300. Then, the transmission/reception unit 410 transmits the information received from the sensor apparatus 200 or the bed terminal 300 to the determination apparatus 500 together with information indicating the transmission source of this information. In other words, the transmission/reception unit 410 transmits the received information to the determination apparatus 500 while making it possible to distinguish whether this information is received from the sensor apparatus 200 or received from the bed terminal 300.

Further, the transmission/reception unit 410 receives the information indicating the result of the agitation determination or the condition determination and/or the like from the determination apparatus 500. Then, the transmission/reception unit 410 transmits the information received from the determination apparatus 500 to the bed terminal 300 or the like.

The determination apparatus 500 is an information processing apparatus that makes the agitation determination or the condition determination according to an acquisition condition of the information. The agitation determination apparatus 500 is set up at a predetermined location such as a nurse station. The determination apparatus 500 is, for example, an information processing apparatus such as a personal computer, a tablet, or a smartphone used by medical staff such as a doctor or a nurse, an on-premise server set up in a hospital or the like, or a cloud server. The determination apparatus 500 may be a combination of the information processing apparatus such as a personal computer, a tablet, or a smartphone, and the server and/or the like. For example, the determination apparatus 500 is a combination of an on-premise server in the hospital and a smartphone, and makes the determination based on the body motion data and/or the vital data by the on-premise server and displays the result and/or notifies the medical staff of the result on the smartphone used by the medical staff. The determination apparatus 500 may be an apparatus different from the above-described examples.

FIG. 5 illustrates an example of the configuration of the determination apparatus 500. Referring to FIG. 5, the determination apparatus 500 includes, for example, an operation input unit 510, a screen display unit 520, a communication OF unit 530, a storage unit 540, and an arithmetic processing unit 550 as main constituent elements thereof.

The operation input unit 510 is configured of an operation input device such as a keyboard and/or a mouse. The operation input unit 510 detects an operation input by the medical staff or the like operating the determination apparatus 500, and outputs it to the arithmetic processing unit 550.

The screen display unit 520 is configured of a screen display device such as an LCD (a Liquid Crystal Display). The screen display unit 520 can display various kinds of information stored in the storage unit 540, such as sensing data 543, score information 544, and result information 545, on the screen according to an instruction from the arithmetic processing unit 550. Note that the screen display unit 520 may be set up at a place remote from the place at which the arithmetic processing unit 550 and the like are set up. For example, only the screen display unit 520 among the configurations included in the determination apparatus 500 may be set up at the nurse station. In this case, the arithmetic processing unit 550 and the like may be set up at a place different from the screen display unit 520, such as a server room.

The communication OF unit 530 is configured of a data communication circuit. The communication OF unit 530 carries out data communication between the determination apparatus 500 and an external apparatus such as the relay apparatus 400 connected via the wireless communication or the like or a mobile terminal carried by the medical staff.

The storage unit 540 is a storage device such as a hard disk or a memory. FIG. 6 illustrates one example of information stored in the storage unit 540. As illustrated in FIG. 6, the storage unit 540 stores processing information required for various kinds of processing by the arithmetic processing unit 550, and a program 546 therein. The program 546 realizes the various kinds of processing units by being read in and executed by the arithmetic processing unit 550. The program 546 is read in from an external apparatus or a recording medium in advance via a data input/output function such as the communication IN unit 530, and is stored in the storage unit 540. Examples of main information stored in the storage unit 540 include an agitation determination-use model 541, a condition determination-use model 542, the sensing data 543, the score information 544, and the result information 545.

The agitation determination-use model 541 is a model for calculating an agitation determination-use score based on the information acquired by the sensor 210. For example, the agitation determination-use model 541 is used when the agitation determination-use score is calculated based on the information acquired via the bed terminal 300. The agitation determination-use model 541 may be used when the agitation determination-use score is calculated based on information acquired via another route. For example, the agitation determination-use model 541 receives information according to at least one of the vital data or the body motion data as an input, and outputs the agitation determination-use score. The agitation determination-use model 541 is, for example, a trained model generated in advance by carrying out machine learning using a support vector machine (SVM), a neural network, or the like in an external apparatus or the like. For example, the machine learning is carried out by using data generated by labeling previously measured vital data and/or body motion data with the presence or absence of the agitation as supervisory data. The agitation determination-use model 541 is acquired from the external apparatus or the like via the communication I/F unit 530 or the like, and is stored in the storage unit 540.

Note that the agitation determination-use model 541 may include a plurality of types of models according to the type of the data. For example, the agitation determination-use model 541 may include a vital model to which the vital data is input, and a body motion model to which the body motion data is input. Further, the agitation determination-use model 541 may include a plurality of models according to the type of the vital data and/or the body motion data, such as the acceleration and the vocal volume.

Further, the agitation determination-use score output from the agitation determination-use model 541 is an index for determining whether the patient is agitated. The agitation determination-use score is, for example, a value from 0 to 1, inclusive. The agitation determination-use score indicates a higher likelihood that the patient is agitated as the value thereof is closer to 1, and indicates a higher likelihood that the patient is not agitated as the value thereof is closer to 0. The agitation determination-use score may be an index expressed using two values that are 1 indicating that the patient is agitated and 0 indicating that the patient is not agitated. The agitation determination-use score may be an index indicating the intensity of the agitation, such as an index set to 2 for strong agitation and 1 for slight agitation.

Further, the data input to the agitation determination-use model 541 may be the vital data and/or the body motion data itself or themselves, or may be various kinds of feature amounts calculated by subjecting the time-series vital data and/or body motion data to processing for converting the time-series data into the feature amount such as averaging processing or differential processing. Further, the agitation determination-use model 541 may be configured to input one type of vital data and/or body motion data or may be configured to input a plurality of types of vital data and/or body motion data.

The condition determination-use model 542 is a model for determining the condition based on the information acquired by the sensor 210. For example, the condition determination-use model 542 is used when the condition is determined based on the information acquired without the intervention of the bed terminal 300 (i.e., transmitted directly from the sensor apparatus 200 to the relay apparatus 400). For example, the condition determination-use model 542 receives the time-series positional information, vital data, body motion data, and/or the like as an input, and outputs information indicating at least one of the positional state, the motion state, the vital state, or the problem state. The condition determination-use model 542 is, for example, a trained model generated in advance by carrying out machine learning using a support vector machine (SVM), a neural network, or the like in an external apparatus or the like. For example, the machine learning is carried out by using data generated by labeling previous time-series positional information, vital data, body motion data, and/or the like with, for example, the presence or absence of the positional state, the motion state, the vital state, and/or the problem state as supervisory data. The condition determination-use model 542 is acquired from the external apparatus or the like via the communication I/F unit 530 or the like, and is stored in the storage unit 540.

Note that the information input to the condition determination-use model 542 is not limited to the positional information. For example, other information directly transmitted from the sensor apparatus 200 to the relay apparatus 400 can be input to the condition determination-use model 542. For example, the condition determination-use model 542 may be a model that receives the vital data and/or the body motion data as an input and outputs the information indicating at least one of the positional state, the motion state, the vital state, the problem state, or the like. Note that the vital data and/or the body motion data input at this time may be data generated by thinning out the vital data, the body motion data, and/or the like acquired by the sensor 210 at a predetermined time interval. Such a model can also be generated in advance by employing a method similar to the above-described method. The condition determination-use model 542 may be configured to input a combination of the above-described examples.

The sensing data 543 includes the information acquired by the sensor 210 of the sensor apparatus 200. For example, the sensing data 543 stores therein at least one of the vital data, the body motion data, the positional information, or the like acquired by the sensor 210 individually for each identification information of the sensor apparatus 200. Note that the sensing data 543 can store the information acquired by the sensor 210 so as to make it possible to identify the route through which the information is acquired.

For example, FIG. 7 illustrates one example of time-series data of the heart rate, which is one type of the vital data. In the case of FIG. 7, an x axis represents time and a y axis represents the heart rate.

The score information 544 includes the agitation determination-use score, which is the index for determining whether the patient is agitated. For example, the score information 544 includes the identification information of the sensor apparatus 200 and the agitation determination-use score in association with each other.

FIG. 8 illustrates one example of the agitation determination-use score calculated by a score calculation unit 553 based on the vital data illustrated in FIG. 7. In the case of FIG. 8, an x axis represents time and a y axis represents the agitation determination-use score. As illustrated in FIG. 8, the agitation determination-use score is expressed by, for example, a value from 0 to 1, inclusive. The agitation determination-use score indicates a higher likelihood that the patient is agitated as the value thereof is closer to 1, and indicates a higher likelihood that the patient is not agitated as the value thereof is closer to 0.

The result information 545 includes the information indicating the result of the determination made by the agitation state determination unit 554 based on the score information 544 or the information indicating the result of the determination made by the condition determination unit 555, and the like. For example, the result information 545 includes the identification information of the sensor apparatus 200 and the information indicating the result of the agitation determination or the result of the condition determination.

The arithmetic processing unit 550 includes a microprocessor such as an MPU and a peripheral circuit thereof. The arithmetic processing unit 550 reads in the program 546 from the storage unit 540 and executes it, thereby causing the above-described hardware and the program 546 to cooperate with each other to realize various kinds of processing units. Examples of main processing units realized by the arithmetic processing unit 550 include a data acquisition unit 551, a specifying unit 552, the score calculation unit 553, the agitation state determination unit 554, and the condition determination unit 555.

The data acquisition unit 551 acquires the body motion data, the vital data, the positional information, the identification information of the sensor apparatus 200, and/or the like transmitted from the relay apparatus 400 via the communication IN unit 530. Then, the data acquisition unit 551 stores the acquired body motion data, vital data, and/or positional information into the storage unit 540 as the sensing data 543 individually for each identification information of the sensor apparatus 200.

The specifying unit 552 specifies which determination processing to perform, the agitation determination by the score calculation unit 553 and the agitation state determination unit 554 or the condition determination by the condition determination unit 555. For example, the specifying unit 552 specifies which determination processing to perform, the agitation determination or the condition determination, according to whether the acquisition condition of the information satisfies a predetermined criterion. For example, the specifying unit 552 specifies to make the agitation determination by the score calculation unit 553 and the agitation state determination unit 554 while the data acquisition unit 551 acquires the information required for the agitation determination. On the other hand, the specifying unit 552 specifies to make the condition determination by the condition determination unit 555 if the data acquisition unit 551 does not acquire at least one of the pieces of information required for the agitation determination. According to such a configuration, the determination processing is supposed to be switched between the agitation determination in which the agitation state determination unit 554 makes the determination, and the determination in which the condition determination unit 555 makes the determination according to the result of the selection specified by the specifying unit 552.

More specifically, for example, the specifying unit 552 specifies the determination processing in the above-described manner based on the data included in the sensing data 543. For example, the specifying unit 552 compares a time stamp of the information acquired via the bed terminal 300, such as the vital data and/or the body motion data, among the pieces of information included in the sensing data 543, and the current time. Then, while the time difference between the time stamp and the current time satisfies a predetermined criterion such as 30 seconds, the specifying unit 552 specifies to make the agitation determination using the score calculation unit 553 and the agitation state determination unit 554. On the other hand, while the time difference between the time stamp and the current time exceeds the predetermined criterion, the specifying unit 552 specifies to make the condition determination by the condition determination unit 555. The specifying unit 552 specifies the determination processing to perform according to the acquisition condition of the information, such as whether the information is normally acquired via the bed terminal 300, in this manner by way of example. Note that the specifying unit 552 may specify to make the agitation determination by the score calculation unit 553 and the agitation state determination unit 554 in addition to or instead of making the condition determination by the condition determination unit 555, if the vital data, the body motion data, and/or the like are/is transmitted through a route not including the bed terminal 300.

Further, for example, the specifying unit 552 can specify the determination processing in the above-described manner based on whether the information acquired via the bed terminal 300, such as the vital data and/or the body motion data included in the sensing data 543, satisfies a predetermined criterion. For example, the specifying unit 552 specifies to make the condition determination by the condition determination unit 555 if the information acquired via the bed terminal 300 satisfies a predetermined criterion such as a criterion that the heart rate stops being measured. On the other hand, until the above-described criterion is satisfied, the specifying unit 552 specifies to make the agitation determination by the score calculation unit 553 and the agitation state determination unit 554.

Further, for example, the specifying unit 552 may specify the determination processing in the above-described manner based on whether the information according to the communication condition received from the bed terminal 300 or the like via the relay apparatus 400 satisfies a predetermined criterion. For example, the specifying unit 552 specifies to make the condition determination by the condition determination unit 555 while the power-saving wireless communication between the sensor apparatus 200 and the bed terminal 300 is determined to be broken based on the information according to the communication condition. On the other hand, the specifying unit 552 specifies to make the agitation determination by the score calculation unit 553 and the agitation state determination unit 554 while the power-saving wireless communication between the sensor apparatus 200 and the bed terminal 300 is determined to be established based on the information according to the communication condition.

Further, the specifying unit 552 may specify which determination processing to perform, the agitation determination or the condition determination, according to a criterion according to the acquisition condition of the information indicating the state or condition of the patient. In other words, the specifying unit 552 may specify which determination processing to perform, the agitation determination or the condition determination, based on a result of the agitation determination or the condition determination acquired when the determination processing is specified. For example, the specifying unit 552 may specify to make the agitation determination when a predetermined condition that the patient returns to the bed is acquired as the information while the patient, for example, goes to a bathroom and the determination apparatus 500 outputs a result of the condition determination.

The specifying unit 552 specifies the determination processing to perform based on the acquisition condition of the information or the like in this manner by way of example. Note that the acquisition condition of the information varies according to, for example, whether the patient stays within a range where the power-saving wireless communication can be carried out with the bed terminal 300. Therefore, it can also be stated that the specifying unit 552 switches the determination processing according to whether the patient leaves (gets off) the bed at which the bed terminal 300 is set up.

Note that the specifying unit 552 may specify which determination processing to perform, the agitation determination or the condition determination, using information different from the acquisition condition of the information. For example, the specifying unit 552 may specify which determination processing to perform, the agitation determination or the condition determination, based on the positional information or the like included in the acquired information. For example, the specifying unit 552 may specify to make the condition determination if the patient is determined to leave the bed based on the positional information while specifying to make the agitation determination if the patient is determined not to leave the bed based on the positional information. Further, the specifying unit 552 may be configured to specify the determination processing based on a combination of some of the above-described plurality of criteria.

The score calculation unit 553 calculates the agitation determination-use score using the agitation determination-use model 541.

For example, the score calculation unit 553 acquires the information acquired via the bed terminal 300 by referring to the sensing data 543. For example, the score calculation unit 553 acquires the vital data including the time-series data of the heart rate like the example illustrated in FIG. 7. Further, the score calculation unit 553 inputs the acquired data to the agitation determination-use model 541, and calculates the agitation determination-use score at each time like the example illustrated in FIG. 8. After that, the score calculation unit 553 stores information indicating the calculated agitation determination-use score into the storage unit 540 as the score information 544. Note that the score calculation unit 553 may calculate the agitation determination-use score based on the vital data, the body motion data, and/or the like acquired without the intervention of the bed terminal 300 when the vital data, the body motion data, and/or the like are/is transmitted directly from the sensor apparatus 200 to the relay apparatus 400.

Note that the score calculation unit 553 may input the time-series data itself to the agitation determination-use model 541, or may input various kinds of feature amounts calculated by subjecting the time-series data to processing for converting the time-series data into the feature amount such as averaging processing or differential processing to the agitation determination-use model 541.

The agitation state determination unit 554 determines whether the patient is in the agitated state based on the agitation determination-use score included in the score information 544. Then, the agitation state determination unit 554 stores the result of the determination into the storage unit 540 as the result information 545. For example, the agitation state determination unit 554 stores information indicating such a result that the patient is determined to be agitated into the storage unit 540 as the result information 545.

For example, the agitation state determination unit 554 includes an agitation determination threshold value to be compared with the agitation determination-use score in advance. Then, the agitation state determination unit 554 makes the determination based on the agitation determination-use score and the agitation determination threshold value. For example, the agitation state determination unit 554 determines that the patient is agitated if the agitation determination-use score is equal to or higher than the agitation determination threshold value. On the other hand, the agitation state determination unit 554 determines that the patient is not agitated if the agitation determination-use score is lower than the agitation determination threshold value.

More specifically, for example, in the case of the agitation determination-use score illustrated in FIG. 8, the agitation determination-use score becomes equal to or higher than the agitation determination threshold value during a period from 22:30 to slightly before 1:00, slightly before 2:00, and around 6:00 as illustrated in FIG. 9. Accordingly, the agitation state determination unit 554 determines that the patient is agitated during these times.

Note that FIG. 9 illustrates an example in which the agitation determination threshold value is set to 0.5. However, the agitation determination threshold value may be a value different from 0.5. The value of the agitation determination threshold value may be set in any manner. For example, the agitation determination threshold value may be determined as appropriate according to, for example, attribute information of the patient, which will be described below.

The condition determination unit 555 estimates the condition of the patient using the condition determination-use model 542.

For example, the condition determination unit 555 acquires the information transmitted directly from the sensor apparatus 200 to the relay apparatus 400 by referring to the sensing data 453. The condition determination unit 555 inputs the acquired information to the condition determination-use model 542, and outputs the information indicating at least one of the positional state, the motion state, the vital state, or the problem state. After that, the condition determination unit 555 stores the output information into the storage unit 540 as the result information 545.

The condition determination unit 555 acquires at least one of the positional information, the vital data, or the body motion data. For example, the condition determination unit 555 acquires the positional data in the form of time-series data. The condition determination unit 555 inputs the acquired positional information to the condition determination-use model 542, and outputs the information indicating the positional state of the patient. Further or alternatively, for example, the condition determination unit 555 acquires the vital data and/or the body motion data. The condition determination unit 555 inputs the vital data and/or the body motion data to the condition determination-use model 542, and outputs the information indicating the motion state, the vital state, and/or the problem state of the patient. Note that the vital data and/or the body motion data may be information generated by thinning out the vital data and/or the body motion data used for the agitation determination at a predetermined time interval. Further or alternatively, the condition determination unit 555 may output information indicating the vital state by inputting the vital data to the condition determination-use model 542, or may output information indicating a further detailed motion state and/or problem state by inputting the vital data and the body motion data to the condition determination-use model 542. In this manner, the condition determination unit 555 may change the condition to determine as appropriate according to the acquired information. Note that the condition determination unit 555 may input the time-series data itself to the condition determination-use model 542, or may input various kinds of feature amounts calculated by subjecting the time-series data to processing for converting the time-series data into the feature amount such as averaging processing or differential processing to the condition determination-use model 542.

Note that the condition determination unit 555 may be configured to make the determination using a method different from the model. For example, the condition determination unit 555 can determine whether a problem has occurred with the patient based on, for example, a duration of stay, which is determined based on the positional information. For example, the condition determination unit 555 can determine that a problem has occurred with the patient if the patient does not move from a predetermined position for a predetermined time based on the positional information. Further, the condition determination unit 555 may make the determination based on the positional information, such as information indicating that the patient is located in an unpermitted room or information indicating that the patient is moving in an unpermitted direction. Further, the condition determination unit 555 may make the above-described determination based on, for example, a movement track of the patient generated based on the positional information. Further, the condition determination unit 555 may be configured to output information indicating the positional state itself according to the position of the patient, such as information indicating the position of the patient.

The notification unit 556 outputs the result of the agitation determination or the result of the condition determination to an external apparatus or the like, for example, when the patient is determined to be agitated by the agitation state determination unit 554, or when the positional state, the motion state, the vital state, the problem state, and/or the like are/is output from the condition determination unit 555. For example, the notification unit 556 can display that the patient is in the agitated state, the information according to the output from the condition determination unit 555, or the like on the screen display unit 520 together with the identification information of the sensor apparatus 200. Further or alternatively, the notification unit 556 can transmit the determination that the patient is in the agitated state, the information according to the output from the condition determination unit 555, or the like, and the identification information of the sensor apparatus 200 to an external apparatus such as the bed terminal 300 relating to this patient or the mobile terminal carried by the medical staff in charge of this patient. The notification unit 556 may issue a notification in a manner different from the above-described example, such as lighting a lamp at an entrance of the room in which the patient is hospitalized.

Note that the notification unit 556 may change the notification method between when the agitation determination is made by the agitation state determination unit 554 and when the condition determination is made by the condition determination unit 555. For example, the notification unit 556 may change the type of the alert between when the agitation state determination unit 554 determines that the patient is agitated and when the condition determination unit 555 determines that a problem has occurred with the patient.

This is an example of the configuration of the determination system 100. Subsequently, examples of operations of the determination apparatus 500 will be described with reference to FIG. 10. Note that the order of the operations of the determination apparatus 500 illustrated in FIG. 10 is one example, and the operations of the determination apparatus 500 are not limited thereto.

The data acquisition unit 551 acquires at least one of the body motion data, the vital data, the positional information, the identification information of the sensor apparatus 200, or the like transmitted from the relay apparatus 400 via the communication I/F unit 530 (step S101).

The specifying unit 552 specifies which determination processing to perform, the agitation determination or the condition determination, according to whether, for example, the information according to the acquisition condition of the information or the communication condition satisfies the predetermined criterion (step S102).

If the criterion is satisfied (NO in step S102), the specifying unit 552 specifies to make the condition determination by the condition determination unit 555 based on the information transmitted from the sensor apparatus 200 to the relay apparatus 400. The condition determination unit 555 determines the condition of the patient based on the information transmitted from the sensor apparatus 200 to the relay apparatus 400 (step S103). The condition determination unit 555 may also make the agitation determination along with the condition determination, if possible. On the other hand, if the criterion is satisfied (YES in step S102), the specifying unit 552 specifies to make the agitation determination by the score calculation unit 553 and the agitation state determination unit 554 based on the information transmitted via the bed terminal 300. The score calculation unit 553 and the agitation state determination unit 554 make the determination about the agitation of the patient based on the vital data, the body motion data, and/or the like acquired via the bed terminal 300 (step S104).

The notification unit 556 outputs the result of the agitation determination or the result of the condition determination, for example, when the patient is determined to be agitated by the agitation state determination unit 554 or when an output is provided from the condition determination unit 555 (step S105). Note that the notification unit 556 may change the notification method between when the patient is determined to be agitated by the agitation state determination unit 554 and when an output is provided from the condition determination unit 555.

In this manner, the determination apparatus 500 includes the specifying unit 552, the agitation state determination unit 554, and the condition determination unit 555. According to such a configuration, the specifying unit 552 can specify which determination processing to perform, the agitation determination by the score calculation unit 553 and the agitation state determination unit 554 or the condition determination by the condition determination unit 555, based on the sensing data 543 or the like. This allows the agitation state determination unit 554 or the condition determination unit 555 to make the determination based on the result of the selection specified by the specifying unit 552 as a result thereof. This result allows an appropriate determination to be made according to the place at which the patient stays.

Note that the positional information may be acquired using a method different from the sensor apparatus 200. For example, the positional information may be acquired by the relay apparatus 400 using an intensity of a beacon or a wireless signal, or the like. In other words, the determination system 100 may be configured in such a manner that the relay apparatus 400 acquires the positional information of the sensor apparatus 200 while the communication is maintained between the relay apparatus 400 and the sensor apparatus 200. In the case where the determination system 100 is configured in this manner, the determination system 100 may be configured in such a manner that the sensor apparatus 200 acquires the vital data and/or the body motion data and the relay apparatus 400 acquires the positional information.

Further, the configuration of the determination apparatus 500 is not limited to the example described with reference to FIG. 5. For example, FIG. 11 illustrates another example of the configuration of the determination apparatus 500. Referring to FIG. 11, the arithmetic processing unit 550 of the determination apparatus 500 can include, for example, attribute information acquisition unit 557.

The attribute information acquisition unit 557 acquires attribute information of a target. For example, the attribute information acquisition unit 557 acquires medical record information of the target from an external apparatus or the like, and acquires attribute information such as an age, a gender, and a paralysis state.

The attribute information acquired by the attribute information acquisition unit 557 can be utilized, for example, when the agitation determination threshold value is adjusted or when the state is determined by the condition determination unit 555. The condition determination unit 555 can adjust, for example, a time taken until the current state is determined to be a state in which a problem has occurred according to whether the patient has hemiplegia. The attribute information acquired by the attribute information acquisition unit 557 may be utilized in a manner different from the above-described examples.

Note that FIGS. 5 and 11 illustrate the example in which the functions as the determination apparatus 500 are realized by one information processing apparatus. However, the functions as the determination apparatus 500 may be realized by, for example, a plurality of information processing apparatuses connected via a network.

### Second Exemplary Embodiment

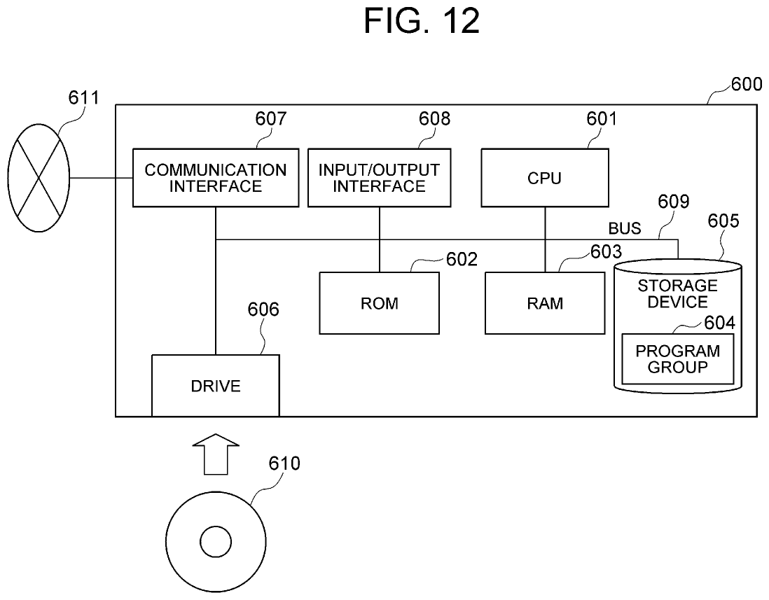

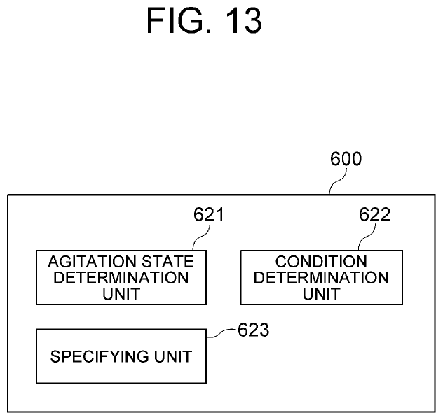

Next, a second exemplary embodiment of the present disclosure will be described with reference to FIGS. 12 and 13. The second exemplary embodiment will be described regarding an overview of the configuration of a determination apparatus 600.

FIG. 12 illustrates an example of the hardware configuration of the determination apparatus 600. Referring to FIG. 12, the determination apparatus 600 has the following hardware configuration as one example.
Central Processing Unit (CPU) 601 (arithmetic unit)
Read Only Memory (ROM) 602 (storage unit)
Random Access Memory (RAM) 603 (storage unit)
Program group 604 to be loaded to the RAM 603
Storage device 605 storing therein the program group 604
Drive 606 that performs reading and writing on a recording medium 610 outside the information processing apparatus
Communication interface 607 connecting to a communication network 611 outside the information processing apparatus
Input/output interface 608 for performing input/output of data
Bus 609 connecting the constituent elements

Further, the determination apparatus 600 can realize functions as an agitation state determination unit 621, a condition determination unit 622, and a specifying unit 623 illustrated in FIG. 13 through acquisition of the program group 604 by the CPU 601 and execution thereof by this CPU 601. Note that the program group 604 is, for example, stored in the storage device 605 or the ROM 602 in advance, and is loaded to the RAM 603 or the like and executed by the CPU 601 as needed. Alternatively, the program group 604 may be provided to the CPU 601 via the communication network 611, or may be stored in the recording medium 610 in advance and read out by the drive 606 and provided to the CPU 601.

Note that FIG. 12 illustrates an example of the hardware configuration of the determination apparatus 600. The hardware configuration of the determination apparatus 600 is not limited to the above-described example. For example, the determination apparatus 600 may be configured of a part of the above-described configuration, such as not including the drive 606.

The agitation state determination unit 621 determines an agitation state of a target based on acquired information.

The condition determination unit 622 determines a condition of the target based on the acquired information.

The specifying unit 623 specifies which to perform, the determination processing performed by the agitation state determination unit 621 or the determination processing performed by the condition determination unit 622 according to an acquisition condition of the information.

In this manner, the determination apparatus 600 includes the agitation state determination unit 621, the condition determination unit 622, and the specifying unit 623. According to such a configuration, the specifying unit 623 can specify which to perform, the determination processing performed by the agitation state determination unit 621 or the determination processing performed by the condition determination unit 622, according to the acquisition condition of the information. This allows an appropriate determination to be made according to whether a patient leaves a predetermined location as a result thereof.

Note that the above-described determination apparatus 600 can be realized by incorporating a predetermined program into this determination apparatus 600. More specifically, a program according to another exemplary embodiment of the present invention is a recording medium recording a program for causing a computer to realize processing including acquiring information and specifying which to perform, determination processing for determining an agitation state of a target or determination processing for determining a condition of the target, according to an acquisition condition of the information.

Further, a determination method realized by the above-described determination apparatus 600 is a method for causing a computer to acquire information and specify which to perform, determination processing for determining an agitation state of a target or determination processing for determining a condition of the target, according to an acquisition condition of the information.

Even the invention of the program (or the recording medium) or the determination method configured in the above-described manner can also bring about advantageous effects equivalent to the above-described determination apparatus 700, thereby achieving the above-described object of the present invention.

### <Supplementary Notes>

The whole or part of the exemplary embodiments disclosed above can be described as, but not limited to, the following supplementary notes. Hereinafter, outlines of the determination apparatus and the like according to the present invention will be described. However, the present invention is not limited to the configurations described below.

### (Supplementary Note 1)

A determination apparatus comprising:
an agitation state determination unit configured to determine an agitation state of a target based on acquired information;
a condition determination unit configured to determine a condition of the target based on the acquired information; and
a specifying unit configured to specify which to perform, the determination processing performed by the agitation state determination unit or the determination processing performed by the condition determination unit, according to an acquisition condition of the information.

### (Supplementary Note 2)

The determination apparatus according to supplementary note 1, wherein
the specifying unit specifies which determination processing to perform, the determination processing performed by the agitation state determination unit or the determination processing performed by the condition determination unit, according to the acquisition condition of information that the agitation state determination unit uses when determining the agitation state.

### (Supplementary Note 3)

The determination apparatus according to supplementary note 1 or 2, wherein
the specifying unit specifies to perform the determination processing in which the condition determination unit makes the determination, in a case where data that the agitation state determination unit uses when determining the agitation state cannot be acquired for a predetermined time.

### (Supplementary Note 4)

The determination apparatus according to any one of supplementary notes 1 to 3, wherein
the specifying unit specifies the determination processing to perform according to a communication condition between a sensor configured to acquire the information and a communication apparatus configured to communicate with the sensor.

### (Supplementary Note 5)

The determination apparatus according to any one of supplementary notes 1 to 4, wherein
the specifying unit specifies which determination processing to perform, the determination processing in which the agitation state determination unit makes the determination or the determination processing in which the condition determination unit makes the determination, according to whether the target leaves a predetermined location.

### (Supplementary Note 6)

The determination apparatus according to any one of supplementary notes 1 to 5, wherein
the agitation state determination unit performs the determination processing based on at least one of vital data or body motion data of the target.

### (Supplementary Note 7)

The determination apparatus according to any one of supplementary notes 1 to 6, wherein
the condition includes any one of a motion state, a vital state, a positional state according to positional information of the target, or a problem state indicating whether a problem has occurred with the target.

### (Supplementary Note 8)

The determination apparatus according to any one of supplementary notes 1 to 7, wherein
the condition determination unit performs the determination processing based on the positional information indicating a position of the target.

### (Supplementary Note 9)

The determination apparatus according to any one of supplementary notes 1 to 8, wherein
the condition determination unit performs the determination processing based on information generated by thinning out the vital data or the body motion data of the target at a predetermined time interval.

### (Supplementary Note 10)

A determination method for causing a computer to:
acquire information; and
specify which to perform, determination processing for determining an agitation state of a target or determination processing for determining a condition of the target, according to an acquisition condition of the information.

### (Supplementary Note 11)

A recording medium recording a program for causing a computer to realize processing including:
acquiring information; and
specifying which to perform, determination processing for determining an agitation state of a target or determination processing for determining a condition of the target, according to an acquisition condition of the information.

Note that the program disclosed in each of the above-described exemplary embodiments and the supplementary notes is, for example, stored in a storage device or recorded in a computer-readable recording medium. For example, the recording medium is a portable medium such as a flexible disk, an optical disk, a magneto-optical disk, or a semiconductor memory.

While the present invention has been described with reference to the exemplary embodiments described above, the present invention is not limited to the above-described exemplary embodiments. The form and details of the present invention can be changed within the scope of the present invention in various manners that can be understood by those skilled in the art.

### REFERENCE SIGNS LIST

100 determination system
200 sensor apparatus
210 sensor
220 determination unit
230 transmission/reception unit
300 bed terminal
310 transmission/reception unit
320 screen display unit
400 relay apparatus
410 transmission/reception unit
420 positional information acquisition unit
500 determination apparatus
510 operation input unit
520 screen display unit
530 communication FP unit
540 storage unit
541 agitation determination-use model
542 condition determination-use model
543 sensing data
544 score information
545 result information
546 program
550 arithmetic processing unit
551 data acquisition unit
552 specifying unit
553 score calculation unit
554 agitation state determination unit
555 condition determination unit
556 notification unit
557 attribute information acquisition unit
700 determination apparatus
701 CPU
702 ROM
703 RAM
704 program group
705 storage device
706 drive
707 communication interface
708 input/output interface
709 bus
710 recording medium
711 communication network
721 agitation state determination unit
722 condition determination unit
723 specifying unit

What is claimed is:

1. A determination apparatus comprising:
at least one memory configured to store processing instructions; and
at least one processor configured to execute the processing instructions to: determine an agitation state of a target based on acquired information of the target;
determine a condition of the target based on the acquired information of the target; and
specify which to perform, the determination processing of the agitation state or the determination processing of the condition, according to an acquisition condition of the information of the target.

2. The determination apparatus according to claim 1, wherein the at least one processor is configured to execute the processing instructions to
specify which determination processing to perform, the determination processing of the agitation state or the determination processing of the condition, according to the acquisition condition of information of the target being used when the agitation state is determined.

3. The determination apparatus according to claim 1, wherein the at least one processor is configured to execute the processing instructions to
specify to perform the determination processing of the condition, in a case where the information of the target used when determining the agitation state cannot be acquired for a predetermined time.

4. The determination apparatus according to claim 1, wherein the at least one processor is configured to execute the processing instructions to
specify the determination processing to perform according to a communication condition between a sensor configured to acquire the information of the target and a communication apparatus configured to communicate with the sensor.

5. The determination apparatus according to claim 1, wherein the at least one processor is configured to execute the processing instructions to
specify which determination processing to perform, the determination processing of the agitation state or the determination processing of the condition, according to whether the target leaves a predetermined location.

6. The determination apparatus according to claim 1, wherein the at least one processor is configured to execute the processing instructions to
determine the agitation state of the target based on at least one of vital data or body motion data of the target.

7. The determination apparatus according to claim 1, wherein
the condition includes at least one of a motion state, a vital state, a positional state according to positional information of the target, or a problem state indicating whether a problem has occurred with the target.

8. The determination apparatus according to claim 1, wherein the at least one processor is configured to execute the processing instructions to
determine the condition of the target based on at least one of the vital data, the body motion data, or the positional information of the target.

9. The determination apparatus according to claim 1, wherein the at least one processor is configured to execute the processing instructions to
determine the condition of the target based on information generated by thinning out at least one of the vital data or the body motion data of the target used to determine the agitation state at a predetermined time interval.

10. A determination method for causing a computer to:
acquire information of a target; and
specify which to perform, determination processing for determining an agitation state of the target or determination processing for determining a condition of the target, according to an acquisition condition of the information of the target.

11. A non-transitory computer readable recording medium recording a program for causing a computer to realize processing including:
acquiring information of a target; and
specifying which to perform, determination processing for determining an agitation state of the target or determination processing for determining a condition of the target, according to an acquisition condition of the information of the target.
